(19) United States
(12) Patent Application Publication
(10) Pub. No.: US 2007/0207223 A1
(43) Pub. Date: Sep. 6, 2007

(54) Means for improving cardiovascular health

(76) Inventors: Maureen A. DiRienzo, Buffalo Grove, IL (US); Cherian George, St. Louis, MO (US); James D. Astwood, Omaha, NE (US); William S. Harris, Sioux Falls, SD (US)

Correspondence Address: MONSANTO COMPANY, 800 N. LINDBERGH BLVD., ATTENTION: GAIL P. WUELLNER, IP PARALEGAL, (E2NA), ST. LOUIS, MO, 63167, US

(21) Appl. No.: 11/712,677

(22) Filed: Mar. 1, 2007

Related U.S. Application Data
(60) Provisional application No. 60/779,135, filed on Mar. 3, 2006.

(51) Int. Cl. A61K 36/899; A61K 36/48; A61K 36/31; G06Q 50/00

(52) U.S. Cl. 424/750; 424/757; 424/776; 424/755; 705/2

(57) ABSTRACT

The present invention is in the fields of lipid metabolism and dietary supplementation and provides methods and compositions to improve heart health of a mammal by orally administering stearidonic acid and related compounds to the mammal. The improved heart health is evidenced by the enrichment of cardiac tissue with eicosapentaenoic acid (20:5, ω3) and docosapentaenoic acid (22:5, ω3) following the administration. Also provided are methods for promoting a stearidonic acid containing product by advertising its heart health benefits.

### CROSS REFERENCE TO RELATED APPLICATIONS

This application claims the benefit of priority to U.S. provisional application Ser. No. 60/779,135, filed Mar. 3, 2006.

### FIELD OF THE INVENTION

The present invention relates generally to the fields of lipid metabolism and dietary supplementation. More particularly, it concerns compositions and methods for controlling or increasing concentrations of eicosapentaenoic acid (EPA) and docosapentaenoic acid (DPA) in cardiovascular system of mammals through the use of genetically engineered seed oils containing stearidonic acid (SDA) and/or its analogs as food ingredients, dietary supplements or pharmaceutical agents.

### BACKGROUND

Omega-3 (ω3) fatty acids are polyunsaturated fatty acids in which a double bond is located between the third and fourth carbon atom from the methyl end of the fatty acid chain. They include, but are not limited to, α-linolenic acid (ALA, 18:3), stearidonic acid (SDA, 18:4), eicosapentaenoic acid (EPA, 20:5), docosapentaenoic acid (DPA, 22:5) and docosahexaenoic acid (DHA, 22:6) and the like.

The therapeutic and preventative benefits of diets enriched in ω3 fatty acids on cardiovascular disease are well documented. For example, epidemiologic studies have supported the idea that people who eat fish containing ω3 fatty acids are at a lower risk for several cardiovascular disease end points than those consuming little or no fish (Dyerberg and Bang (1979) Haemostasis 8: 227-33; Kromhout et al. (1985) N. Engl. J. Med. 312: 1205-09). Several important studies reported recently have solidified the view that dietary ω3 fatty acids are cardioprotective. For example, analyses in the GISSI-Prevenzione trial indicated that patients surviving a recent myocardial infarction had a significantly lower risk of cardiovascular death if their diets were supplemented with about 1 g/d of ω3 fatty acids (Investigators GISSI-Prevenzione (1999) Lancet 354: 447-55). Additionally, in the Nurses' Health Study, women without prior cardiovascular disease had a lower risk of coronary heart disease with the intake of fish or ω3 fatty acids (Hu et al. (2002) J. Am. Med. Assoc. 287: 1815-21). A direct link between tissue concentrations of ω3 fatty acids and cardiovascular disease risk was also reported in a prospective, nested case-control analysis of men enrolled in the Physicians' Health Study in which blood concentrations of ω3 fatty acids were inversely related to risk of sudden death among men without prior evidence of cardiovascular disease (Albert et al. (2002) N. Engl. J. Med. 346: 1113-8).

As indicated above, it is now well-established that the ω3 fatty acids can reduce the risk of heart attacks and deaths due to heart disease. The means by which these omega-3 fatty acids exert these effects is not entirely elucidated, but it is hypothesized that the presence of these omega-3 fatty acids in the membranes of heart cells makes them resistant to ventricular fibrillation, the uncoordinated, arrhythmic contraction of heart cells which precedes heart attacks.

The cardiovascular health benefits of ω3 fatty acids are largely attributed to EPA and DHA, whose presence in tissues is directly related to their dietary intake. Thus, the American Heart Association recently published a Scientific Statement that individuals at risk for coronary heart disease would benefit from the consumption of fish or fish oils, which provide EPA and DHA. However, the individuals at risk for coronary heart disease and the public in general have been slow in supplementing their diets with fish or fish oil. This is in part due to dietary habit and in part due to concerns about environmental contaminants such as heavy metals, methylmercury, and organochlorides in fish or fish oil (Kris-Etherton et al. (2002) Circulation 106: 2747-57).

It is imperative to find alternative sources with which to supplement the human diet in order to provide sufficient amount of EPA and DHA in the cardiovascular system. One such alternative source would be a vegetable oil that contains one of the precursors leading to EPA and DHA. For example, the ω3 fatty acid ALA was contemplated as such a source because it can be converted to SDA by a Δ6-desaturase. SDA can then be converted into EPA through the sequential action of an elongase and a Δ5-desaturase. Despite its abundancy in widely consumed commercial oils of plant origin such as canola and soybean oils, ALA from regular dietary intake was proven ineffective in raising the tissue concentrations of EPA and DHA. This is likely due to the inefficiency of the Δ6-desaturase-catalyzed step (Kelley et al. (1993) Lipids 28: 533-7). SDA is the product of the reaction on ALA catalyzed by the Δ6-desaturase. Thus, by providing SDA directly in the diet, one could bypass the rate-limiting step and provide the substrate for the synthesis of EPA and DHA. Ingestion of vegetable oils rich in SDA may lead to an enrichment of tissues with longer-chain polyunsaturated fatty acids such as EPA, thus mimicking the beneficial effects typically associated with the consumption of fish or fish oils.

A number of vegetable oils have been reported to contain SDA in sufficient quantity. For example, there are naturally occurring seeds of the Boraginaceae family with SDA content of about 17% of total fatty acids, although currently these are wildflower seeds and not cultivated oilseeds (U.S. Pat. No. 6,340,485; Velasco and Goffman (1999) Phytochemistry 52: 423-6). Additionally, a dietary oil extracted from seeds of the Echium plantagineum plant (Echium oil) is also found to contain SDA in substantial quantities (approx. 12.5% of total fatty acids). SDA-containing vegetable oils are also found in seed oils of genetically engineered crops. For example, canola (U.S. Pat. No. 6,459,018), corn (PCT Publication WO 2005102310), and soybean (PCT Publication WO 2005021761) have been genetically engineered to produce seed oils containing SDA of over 10% by weight of total fatty acids.

Work has been performed on the dietary supplementation of SDA or SDA-containing oils. For example, dietary supplementation with SDA has been shown to increase the concentrations of EPA and DPA in the phospholipid fractions of erythrocytes and plasma (James et al. (2003) Am. J. Clin. Nutr. 77: 1140-5). A dietary supplementation with Echium oil also increased the tissue concentrations of EPA and DPA in plasma and neutrophils (Surette et al. (2004) J. Nutr. 134: 1406-11). However, many questions remain to be answered including, but not limited to, the conversion of SDA into longer chain polyunsaturated fatty acids such as EPA, DPA, and DHA in the cardiac tissue, potential of SDA containing compounds as vehicles of imparting heart health benefits, and means of delivering said compounds. These and other questions are at least partially addressed by the present disclosure.

### SUMMARY OF THE INVENTION

The present invention is directed to dietary or pharmaceutical means that increase concentrations of heart-health-promoting polyunsaturated fatty acids in the cardiovascular system of mammals. In one embodiment of the invention, a composition is provided wherein said composition comprising a compound containing a SDA moiety for enriching cardiac tissues of mammals with EPA and DPA. This compound can be provided as a free fatty acid, a fatty acyl ester, a monoglyceride, a diglyceride, a triglyceride, an ethyl ester, a phospholipid, a steryl ester, a sphingolipid, or a combination of these.

In one embodiment of the invention, the composition is provided as an endogenous seed oil from a plant that is genetically engineered to produce SDA.

A further aspect of the invention is a food product comprising from 0.01% to 99%, preferably 10 to 50%, more preferably 20% to 40% by weight of a composition of the invention.

Further, the invention relates to a method of enriching cardiovascular tissues of mammals with EPA and DPA comprising orally administering a nutritionally or therapeutically effective amount of a compound containing a SDA moiety. The compound can be provided as a free fatty acid, a fatty acyl ester, a monoglyceride, a diglyceride, a triglyceride, an ethyl ester, a phospholipid, a steryl ester, a sphingolipid, or a combination of these. Administration of this compound can be performed at doses on a human equivalent basis, for example, from about 0.1 mg/kg/day to 2 g/kg/day, preferably from about 1 mg/kg/day to about 1 g/kg/day, and more preferably from about 20 mg/kg/day to about 500 mg/kg/day.

Another aspect of the invention is to provide a method for promoting a product as improving the heart health of a mammal by advertising and/or labeling the product as containing SDA. The product can be a food product, a dietary supplement, or a pharmaceutical product.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. RBC Omega-3 Fatty Acid Content—2 Weeks

FIG. 2. RBC Omega-3 Fatty Acid Content—4 Weeks

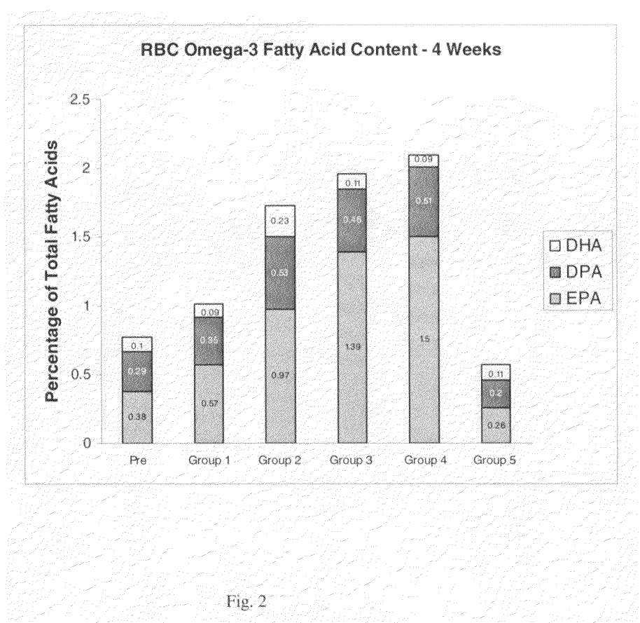

FIG. 3. RBC Omega-3 Fatty Acid Content—8 Weeks

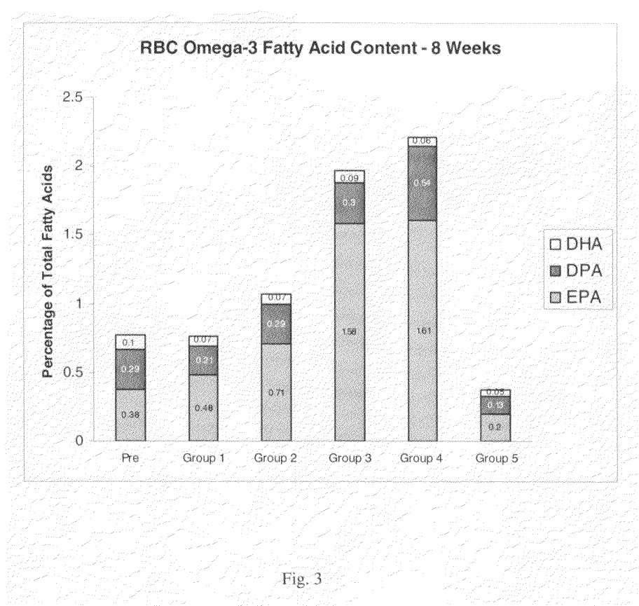

FIG. 4. RBC Omega-3 Fatty Acid Content—12 Weeks

FIG. 5. RBC Omega-3 Fatty Acid Content—21.4 mg/kg/day SDA

FIG. 6. RBC Omega-3 Fatty Acid Content—64.2 mg/kg/day SDA

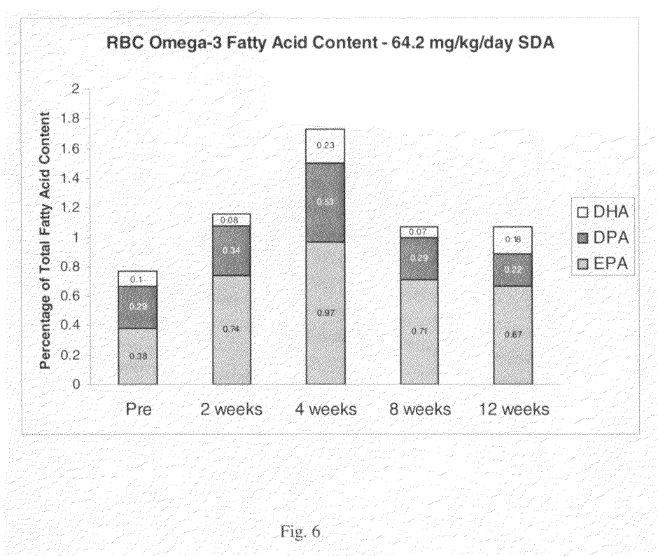

FIG. 7. RBC Omega-3 Fatty Acid Content—192.9 mg/kg/day SDA

FIG. 8. RBC Omega-3 Fatty Acid Content—42.9 mg/kg/day EPA

FIG. 9. RBC Omega-3 Fatty Acid Content—Sunflower Oil

FIG. 10. Heart Omega-3 Fatty Acid Content—4 Weeks

FIG. 11. Heart Omega-3 Fatty Acid Content—8 Weeks

FIG. 12. Heart Omega-3 Fatty Acid Content—12 Weeks

FIG. 13. Heart Omega-3 Fatty Acid Content—21.4 mg/kg/day SDA

FIG. 14. Heart Omega-3 Fatty Acid Content—64.2 mg/kg/day SDA

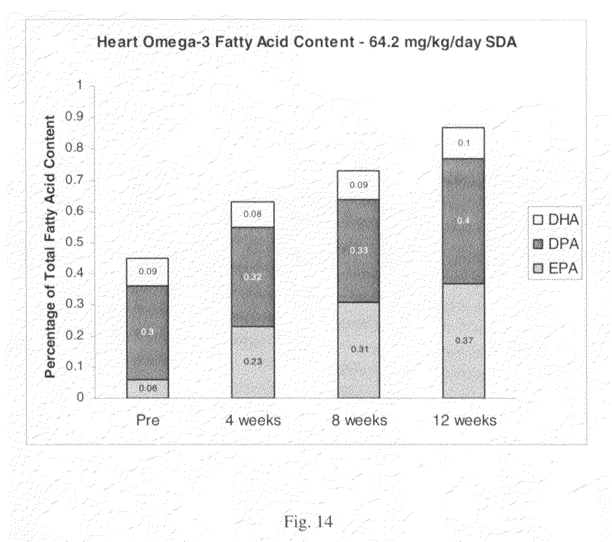

FIG. 15. Heart Omega-3 Fatty Acid Content—192.9 mg/kg/day SDA

FIG. 16. Heart Omega-3 Fatty Acid Content—42.9 mg/kg/day EPA

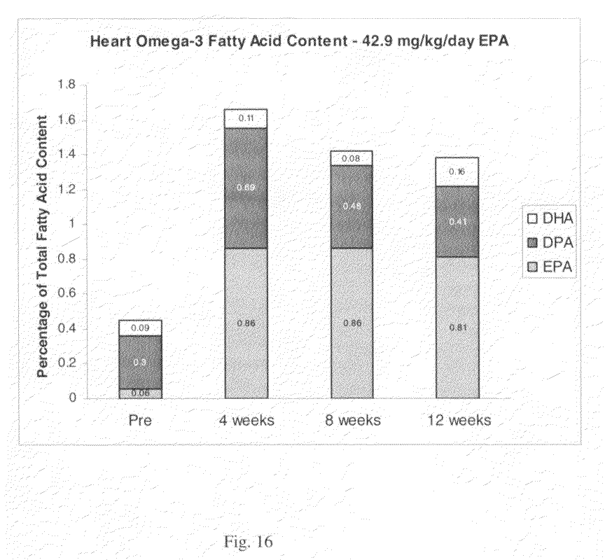

FIG. 17. Heart Omega-3 Fatty Acid Content—Sunflower Oil

### DETAILED DESCRIPTION OF THE INVENTION

Stearidonic acid (SDA) is an 18-carbon omega-3 fatty acid with four double bonds in the all cis 6, 9, 12, and 15 positions. It is present in the food supply in milligram/serving amounts, primarily from fish sources. Current dietary sources of other omega-3 fatty acids include fish and fish oil, which provide eicosapentaenoic acid (EPA) and docosahexaenoic acid (DHA), and oilseeds and nuts which provide alpha-linolenic acid (ALA). Typical dietary intakes of EPA and DHA are well below recommended intakes because fish, especially omega-3 rich fatty fish, are not widely or frequently consumed. Health authorities have recognized that EPA and DHA are associated with heart health effects; specifically, consumption of these omega-3 fatty acids has been shown to reduce the risk of sudden fatal heart attacks.

The typical dietary intake of ALA has been deemed adequate to meet basic nutritional needs, but ALA has not been shown to exert the heart health effects associated with EPA and DHA. ALA is mostly β-oxidized or metabolized to other products of fatty acid metabolism; very little is converted in the body to EPA and DHA. This is because the first enzyme in the bioconversion of ALA to EPA and DHA, Δ6 desaturase, is rate-limiting. SDA is the product of the reaction on ALA catalyzed by the Δ6-desaturase. Thus, by providing SDA directly in the diet, one bypasses the rate-limiting step and provides the substrate for the synthesis of EPA and DHA.

As indicated above, it is now well-established that the omega-3 fatty acids, EPA and DHA, as provided in fish and fish oil, can reduce the risk of heart attacks and deaths due to heart disease. The means by which these omega-3 fatty acids exert these effects is not entirely elucidated, but it is hypothesized that the presence of these omega-3 fatty acids in the membranes of heart cells makes them resistant to ventricular fibrillation, the uncoordinated, arrhythmic contraction of heart cells which precedes heart attacks.

It is therefore one objective of the present invention to provide a composition wherein said composition comprising a compound containing a SDA moiety for enriching cardiac tissues of mammals with EPA and DPA. This compound can be provided as a free fatty acid, a fatty acyl ester, a monoglyceride, a diglyceride, a triglyceride, an ethyl ester, a phospholipid, a steryl ester, a sphingolipid, or a combination of these. The preparation of a composition that contains a compound with a SDA moiety alone or in combination with other supplements will be known to those of skill in the art in light of the present disclosure. Typically, such compositions can be prepared as liquids or capsules; solid forms or suspensions; the preparations can also be emulsified.

The compositions of the invention are preferably suitable for use in a food product. The compositions may be consumed themselves, but they are typically incorporated into a food product or a nutritional to supplement before consumption. Therefore, a further aspect of the invention is a food product comprising from 0.01% to 99%, preferably 10 to 50%, more preferably 20% to 40% by weight of a composition of the invention. Food products that may be utilized to practice the present invention include, but are not limited to: beverages, (including soft drinks, carbonated beverages, ready to mix beverages and the like), infused foods (e.g. fruits and vegetables), sauces, condiments, salad dressings, fruit juices, syrups, desserts (including puddings, gelatin, icings and fillings, baked goods, and frozen desserts such as ice creams and sherbets), chocolates, candies, soft frozen products (such as soft frozen creams, soft frozen ice creams and yogurts, soft frozen toppings, such as dairy or non-dairy whipped toppings), oils and emulsified products (such as shortening, margarine, mayonnaise, butter, cooking oil, and salad dressings), prepared meats (such as sausage), intermediate moisture foods, (e.g. rice and dog foods) and the like.

Food products can be enriched in a SDA-containing composition by conventional methods such as obtaining the composition and evenly distributing it throughout the food product, to which it is added by dissolution, or by suspension, or in an emulsion. For example, the composition can be dissolved in an edible solubilizing agent, or can be mixed with an edible solubilizing agent, an effective amount of a dispersant, and optionally, an effective amount of an antioxidant. Examples of useful antioxidants include, but are not limited to, tocopherols, such as α-tocopherol, ascorbic acid, inexpensive synthetic antioxidants, and mixtures thereof. Food products may also be prepared from transgenic plants engineered for increased SDA. Examples of such plants having increased SDA that may be used with the invention are described in U.S. Pat. No. 6,459,018, the disclosure of which is incorporated herein by reference.

Effective carriers for preparing emulsions or suspensions include water, alcohols, polyols and mixtures thereof. Examples of useful dispersants include, but are not limited to, lecithin, other phospholipids, sodium lauryl sulfate, fatty acids, salts of fatty acids, fatty acid esters, other detergent-like molecules, and mixtures thereof. Alternatively, the food products can be made by a method comprising obtaining SDA-containing composition and mixing it with an edible solubilizing agent and an effective amount of a dispersant. Again, the edible solubilizing agent can include, but is not limited to, monoglycerides, diglycerides, triglycerides, vegetable oils, tocopherols, alcohols, polyols, or mixtures thereof, and the dispersant can include, but is not limited to, lecithin, other phospholipids, sodium lauryl sulfate, fatty acids, salts of fatty acids, fatty acid esters, other detergent-like molecules, and mixtures thereof.

A further embodiment of the invention relates to a method of enriching cardiac tissues of mammals with EPA and DPA comprising orally administering a nutritionally or therapeutically effective amount of a compound containing a SDA moiety. The compound can be provided as a free fatty acid, a fatty acyl ester, a monoglyceride, a diglyceride, a triglyceride, an ethyl ester, a phospholipid, a steryl ester, a sphingolipid, or a combination of these. Administration of this compound can be performed at doses on a human equivalent basis, for example, from about 0.1 mg/kg/day to 2 g/kg/day, preferably from about 1 mg/kg/day to about 1 g/kg/day, and more preferably from about 20 mg/kg/day to about 500 mg/kg/day.

The phrase “nutritionally effective” as used herein indicates the capability of an agent to affect the structure or function of the body or to reduce the risk for disease. The phrase “therapeutically-effective” as used herein indicates the capability of an agent to prevent, or improve the severity of, the disorder, while avoiding adverse side effects typically associated with alternative therapies. The phrase “therapeutically-effective” is to be understood to be equivalent to the phrase “effective for the treatment or prevention,” and both are intended to qualify, e.g., the amount of stearidonic acid used in the methods of the present invention which will achieve the goal of improvement in the severity of a disorder or preventing the disorder while avoiding adverse side effects typically associated with alternative therapies.

For pharmaceutical use (human or veterinary), the compositions are generally administered orally but can be administered by any route by which they may be successfully absorbed, e.g., parenterally (i.e. subcutaneously, intramuscularly or intravenously), rectally or vaginally or topically, for example, as a skin ointment or lotion. The compositions of the present invention may be administered alone or in combination with a pharmaceutically acceptable carrier or excipient. Where available, gelatin capsules may be a preferred form of oral administration. Dietary supplementation as set forth above can also provide an oral route of administration.

The amount of the compound containing a SDA moiety that is nutritionally or therapeutically effective depends on multiple factors, such as the prior nutritional and physiological status of the consumer, the seriousness of heart disorder being treated, dietary habits of a patient, the age of the patient, presence of additional conditions, etc. A person who consumes relatively small amounts of the compound in his/her normal diet will need a greater amount than one who typically consumes a greater amount of SDA. One skilled in the art would know how to determine the therapeutically effective amount for a patient based on these considerations.

A further aspect of the invention relates to a business method for promoting the sale of a product by advertising the product as containing SDA and improving heart health of a mammal following ingestion of the product. The product is preferably a food product, a nutraceutical product, or a pharmaceutical product. By informing the public about the heart health benefits as disclosed herein, one can realize, for example, the benefit of increased sales of the product. Traditional advertising channels, including but not limited to, radio, TV and printed publications, can be employed for this purpose. Any new and emerging electronic media for advertising are also contemplated in this context.

The following example is included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention.

### EXAMPLE

### Background and Objectives

There are numerous health benefits associated with the dietary long chain n-3 fatty acids eicosapentaenoic acid (EPA) and docosahexaenoic acid (DHA). Specifically, both EPA and DHA, which are commonly found in fish and fish oils, have been widely-implicated as being beneficial in the reduction of risk of cardiovascular disease (Ismail (2005) Frontiers in Bioscience 2005, 10: 1079-88). Negative consumer perceptions involving possible contaminants in fish, the poor palatability of fish oils and the general dietary preferences of the population in landlocked areas limit the feasibility of dietary n-3 fatty acid supplementation using common sources (Verbeke et al. (2005) Public Heath Nutrition 8(4): 422-9). Stearidonic acid (SDA), a viable alternative to long chain n-3 fatty acids found in fish, is derived from plant sources and can be incorporated into common foods. SDA has been previously shown to be effectively converted into EPA and DPA in erythrocytes and plasma when administered via the human diet (James et al., 2003). In order to further evaluate the efficacy of SDA with regard to the enrichment of cardiac tissue with EPA, DPA and DHA, a dietary study was initiated to determine if feeding SDA would result in enrichment of cardiac tissue with EPA, DPA and DHA in beagle dogs for a period of 3 months.

### Study Design

The test article, SDA, was administered in the diet once daily, 7 days per week, for up to 90 days to three groups (Groups 1-3) of male beagle dogs. The reference article, eicosapentaenoic acid (EPA), was administered to Group 4 on the same dosing regimen. Both SDA and EPA were supplied as ethyl esters. The control article, food grade high oleic sunflower oil (SFO), was administered to Group 5 on the same dosing regimen. SFO was also added to food of groups 1 through 4 so that all animals received a similar volume of oil per kg body wt. A dietary supplement, vitamin E, was added to all diets. Dosage levels were 21.4, 64.2 and 192.9 mg/kg/day SDA, 42.9 mg/kg/day EPA and were calculated for each animal based on body weight for Groups 1, 2, 3 and 4, respectively. Each group consisted of 15 males. Five animals/group were scheduled for each interim necropsy (study weeks 4 and 8) and the primary necropsy at the end of the 12-week treatment period. In addition, five animals were euthanized prior to randomization and test article administration to establish baseline levels of fatty acids (pretest necropsy).

The animals were observed twice daily for mortality and moribundity. Clinical examinations were performed daily, and detailed physical examinations were performed weekly. Individual body weights were recorded weekly. Food consumption was recorded daily and reported weekly. Blood samples for analysis of fatty acids were collected from five dogs scheduled for the pretest necropsy and from all surviving dogs during study weeks 2, 4, 8 and 12. All animals were euthanized, and in addition to liver and kidney sections, two samples of heart tissue were collected, one set was analyzed for fatty acid analysis and the other sample was retained for microscopic analysis at the scheduled necropsies. Sections from the heart, liver and kidney were examined microscopically from five animals during pretest and from 5 animals/group (Groups 3-5 only) at the study week 12 necropsy.

### Fatty Acid Analysis

Blood samples were collected from the 5 dogs selected for the pretest necropsy and from all surviving dogs at study weeks 2, 4, 8 and 12. The samples were collected from the dog's jugular vein into tubes containing EDTA prior to the feeding/dosing regimen. The red blood cells (RBC) were separated from the plasma by centrifugation at 1500× for approximately 20 minutes at 4° C. The plasma was transferred to polypropylene tubes and stored at approximately −70° C. for future analysis. The buffy coat was removed from the packed RBCs, and the RBCs were divided approximately equally into two tubes.

Tissue preparation, lipid extraction and analysis were conducted according to a conventional protocol. Briefly, heart tissues were first lyophilized overnight, and then pulverized by grinding between two ground glass slides. The ground tissue was suspended in saline and subjected to 10-15 seconds of sonication. Lipids were extracted with methanol and methylene chloride, and the solvent evaporated under nitrogen. Thawed RBCs were extracted with isopropanol and hexane. After centrifugation of the stroma, the solvent was transferred and evaporated under nitrogen. Phospholipids extracted from heart and RBC samples were methylated with BF₃, at 100° C. for 10 minutes. These conditions transmethylate glycerophospholipid FAs but not sphingolipid FAs. After heating, all samples were extracted with hexane and water. The hexane layer was removed, dried under nitrogen, and the FA methyl esters reconstituted in hexane for analysis by flame ionization gas chromatography. FAs were identified by comparison with known standards, and FA compositions were reported as weight percent of total FA.

### Macroscopic Examination

Animals were euthanized by an intravenous injection of sodium pentobarbital followed by exsanguinations. Two samples of approximately 200 mg of heart tissue from the left ventricle were collected, rinsed in chilled saline, wrapped in aluminum foil, flash frozen in liquid nitrogen, and stored at approximately −70° C. The samples were analyzed for pathologic abnormalities.

### Microscopic Examination

The heart, liver, and kidney sections were placed in 10% neutral-buffered formalin. The tissues were trimmed and placed into paraffin blocks, sectioned at 4 to 8 microns, mounted on glass microscope slides, and stained with hematoxylin and eosin. The samples were analyzed for pathologic abnormalities.

### Statistical Analysis

Body weight, body weight change, and food consumption data were subjected to a one-way ANOVA to determine intergroup differences. If the ANOVA revealed statistically significant (p<0.05) intergroup variance, Dunnett's test (Dunnett, 1964) was used to compare the test article-treated groups to the control group.

### Results

All animals survived to the scheduled necropsies. There were no test article-related clinical findings or effects on body weight or food consumption. There were no microscopic findings attributed to test article administration.

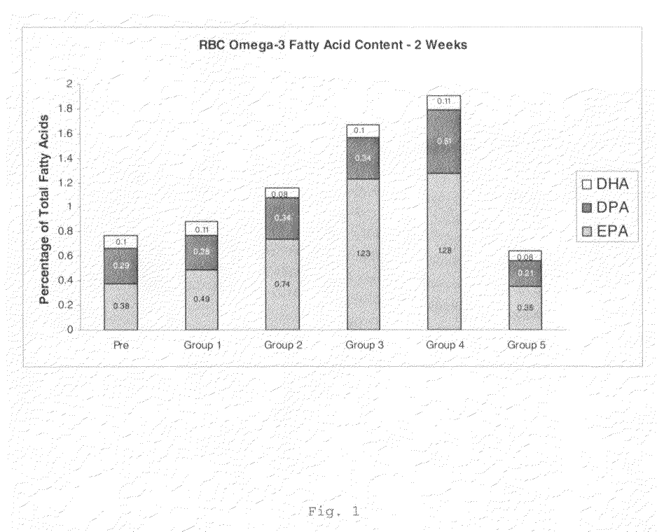

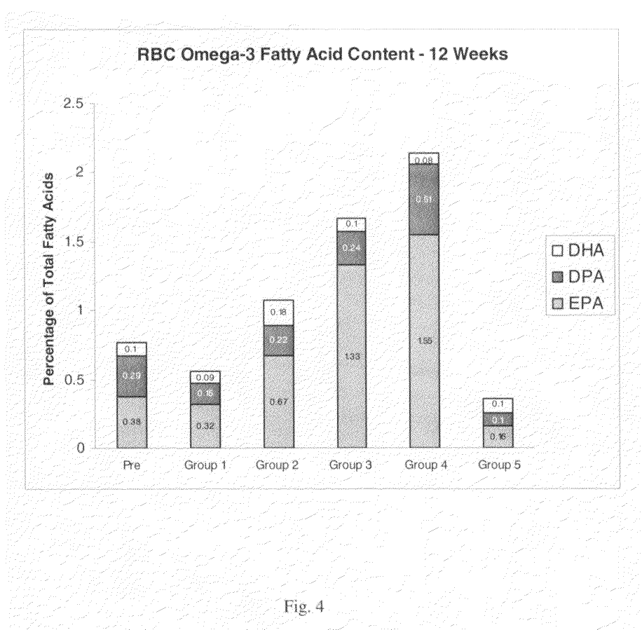

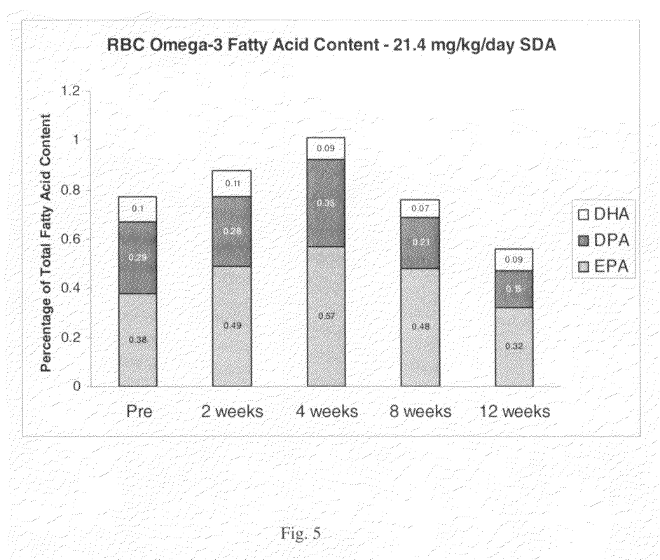

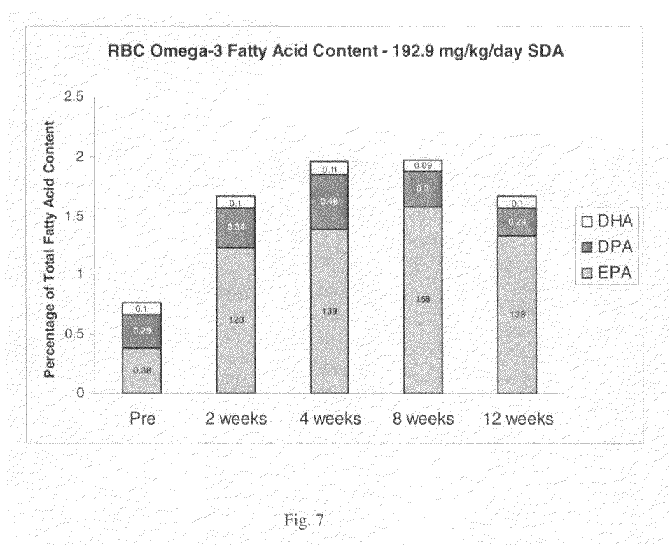

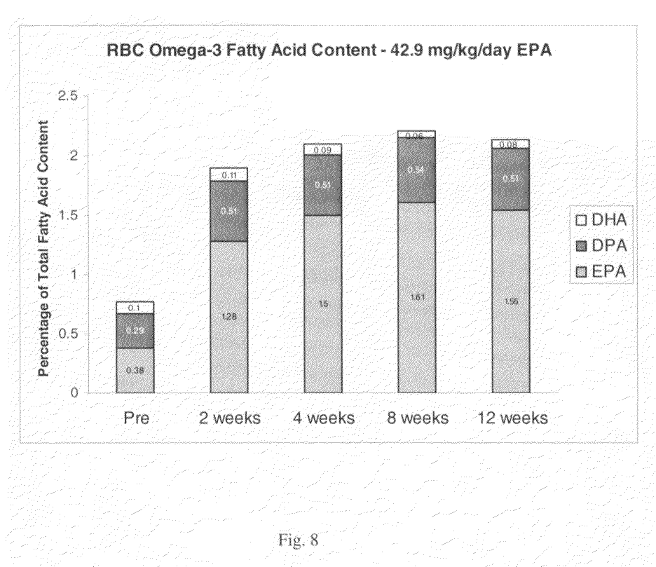

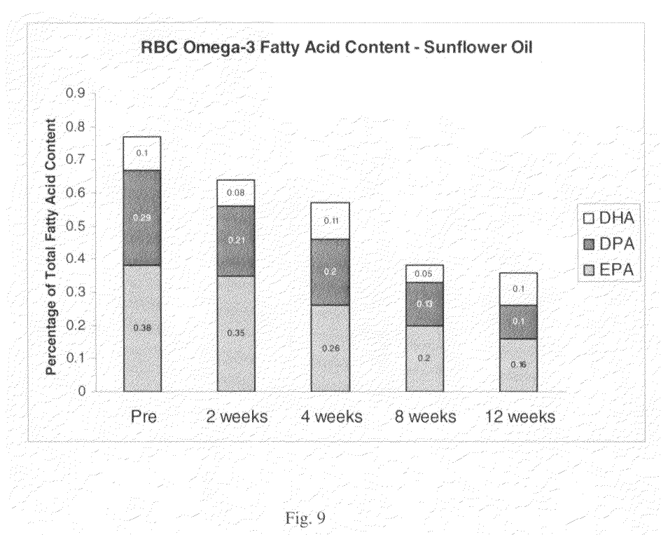

Omega-3 fatty acid content found in the red blood cells (RBC) of SDA-treated dogs was shown to be increased in a dose-dependent manner at study weeks 2, 4, 8 and 12. The RBC omega-3 fatty acid content of EPA-treated dogs (reference article) was approximately 3-10 fold higher than SFO-treated dogs (negative control) between study weeks 2 and 12 (FIGS. 1 and 4). Treatment with 21.4 mg/kg/day SDA showed an overall increase in total omega-3 fatty acid content (RBC) over pretreatment levels as early as study week 2, peaking at approximately 4 weeks and decreasing thereafter (FIG. 5). Treatment with 192.9 mg/kg/day SDA (Group 3) showed an overall increase in total omega-3 fatty acid content (RBC) over pretreatment levels as early as study week 2 and remained elevated (approximately 2-fold over pretreatment) through 12 weeks (FIG. 7). While slightly lower, the overall increase in omega-3 fatty acid content (RBC) in the 192.9 mg/kg/day SDA group over time was comparable to the increase seen in the 42.9 mg/kg/day EPA group (FIGS. 7 and 8). Treatment with sunflower oil (negative control) did not show an increase in RBC omega-3 fatty acid content over time, but rather an almost 50% decrease between study weeks 2 and 12 (FIG. 9).

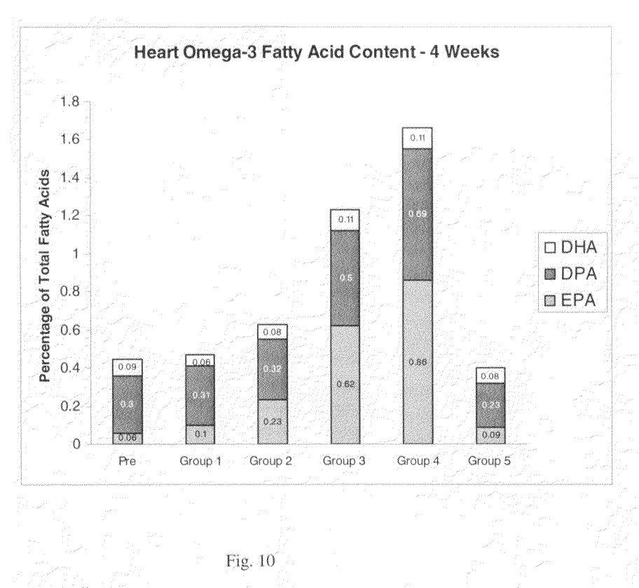

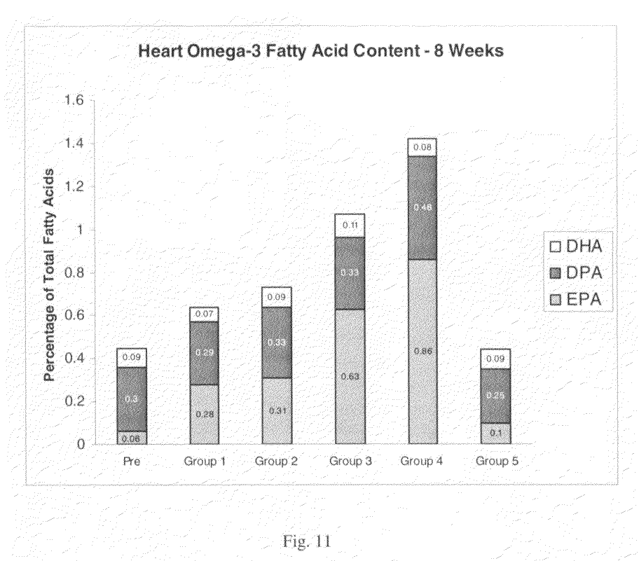

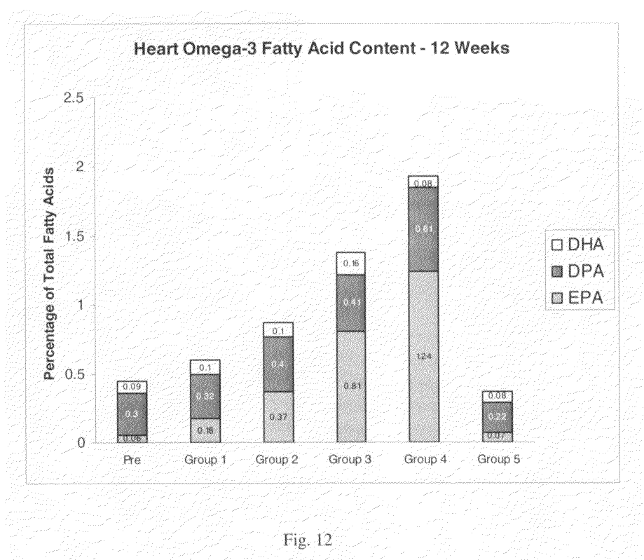

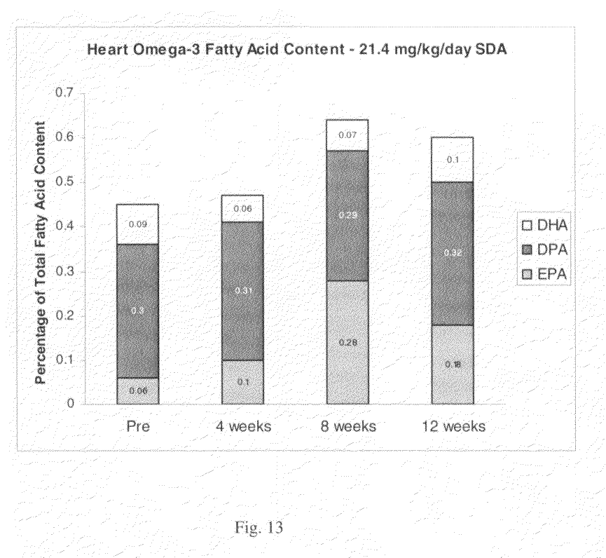

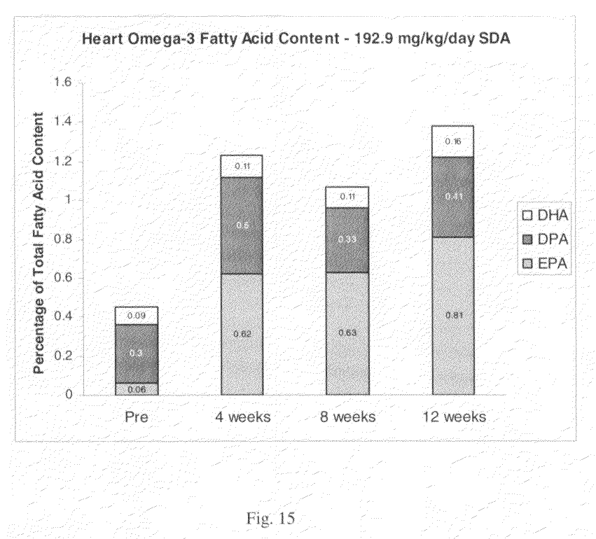

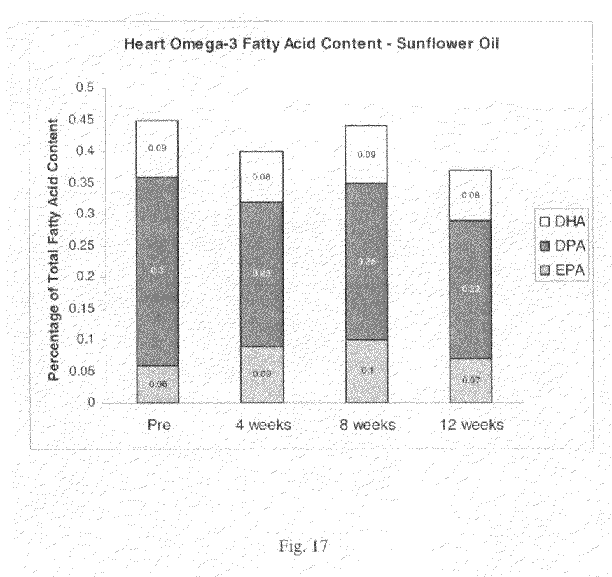

Similar to the RBCs, omega-3 fatty acid content in the heart tissue of SDA-tested dogs was shown to be increased in a dose-dependent manner at study weeks 4, 8 and 12 (FIGS. 10-12). Omega-3 fatty acid content in the heart tissues of EPA-treated dogs (reference article) was approximately 3-5 fold higher than sunflower oil-treated dogs (negative control) between study weeks 4 and 12 (FIGS. 10-12). Treatment with 21.4 mg/kg/day SDA showed a negligible increase in heart omega-3 fatty acid content over pretreatment levels at study week 4 and peaked (approximately 1.5 fold over pretreatment levels) at study week 8. Similar results were obtained for the 64 mg/kg group (FIG. 13). Treatment with 192.9 mg/kg/day showed a more dramatic increase in heart omega-3 fatty acid content, approximately 3 fold higher than pretreatment levels at study week 12 (FIG. 15). Treatment with sunflower oil (negative control) did not show any significant changes in heart omega-3 fatty acid content between study weeks 4 and 12 (FIG. 17).

### CONCLUSIONS

Stearidonic acid (SDA) administered via the diet daily for 12 consecutive weeks to male beagle dogs was effectively converted into EPA and DPA and was found incorporated into cardiac and red blood cell membranes. Furthermore, administration of SDA up to 192.9 mg/kg/day for 12 weeks did not show any adverse clinical effects and did not cause any significant microscopic changes to heart, liver and kidney tissues. This is the first study to demonstrate conclusively that oral administration of SDA to a mammal can enrich cardiac tissue with EPA and DPA, thereby improving heart health of the mammal.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

All publications and patent applications cited in this specification are herein incorporated by reference as if each individual publication or patent application was specifically and individually indicated to be incorporated by reference.

What is claimed is:

1. A method of feeding a mammal comprising orally administering an amount of an endogenous seed oil of a genetically engineered crop, wherein said seed oil has sufficient stearidonic acid to enrich cardiac tissue or red blood cells of the animal with eicosapentaenoic acid (20:5, ω3) and docosapentaenoic acid (22:5, ω3)

2. The method of claim 1, wherein said mammal is a human.

3. The method of claim 1, wherein said mammal is a companion animal.

4. The method of claim 1, wherein said seed oil administered comprises from about 1 mg/kg/day to about 5000 mg/kg/day on a human equivalent basis.

5. The method of claim 4, wherein said seed oil administered comprises from about 20 mg/kg/day to about 2000 mg/kg/day.

6. The method of claim 1, wherein said seed oil is selected from the group consisting of soybean oil, corn oil, and canola oil.

7. The method of claim 1, wherein said seed oil is delivered as a pharmaceutical agent.

8. The method of claim 1, wherein said seed oil is present in a food product selected from the group consisting of bakery products, oil-based products, dairy products, infant formulas, and non-dairy beverages.

9. The method of claim 8, wherein said food product is selected from bread, biscuits or cookies, snack bars, milk, reconstitutable milk products, spreads, salad dressings, ice cream and fruit juice.

10. A method of treating coronary heart disease or dyslipidemia of a mammal comprising administering to a mammal a therapeutically effective amount of an endogenous seed oil of a genetically engineered crop, wherein said endogenous seed oil contains stearidonic acid of at least 5% of total fatty acids.

11. The method of claim 10, wherein said coronary heart disease comprises arrhythmia, thrombosis, hypertension, arteriosclerosis/atherosclerosis, and post myocardial infarction.

12. A method for promoting a product as improving heart health of a mammal comprising the step of advertising or labeling said product as containing stearidonic acid.

13. The method of claim 12, wherein promoting a product comprises attempting to increase sale of the product.

14. The method of claim 12, wherein said product is selected from the group consisting of a food product, a dietary supplement product, and a pharmaceutical product.

15. The method of claim 12, wherein said advertising is achieved by means comprising product labels, printed publication, radio, television, and other electronic media.
